(19) United States
(12) Patent Application Publication
(10) Pub. No.: US 2006/0052474 A1
(43) Pub. Date: Mar. 9, 2006

(54) Sound-insulating material and method for the production thereof

(76) Inventor: Hans Rudolf Czerny, Swisttal (DE)

Correspondence Address: WILLIAM COLLARD;COLLARD & ROE, P.C., 1077 NORTHERN BOULEVARD, ROSLYN, NY, 11576, US

(21) Appl. No.: 10/523,663

(22) PCT Filed: Aug. 11, 2003
(86) PCT No.: PCT/EP03/08905

§ 371 Date: Feb. 4, 2005

(30) Foreign Application Priority Data
Aug. 13, 2002 (DE) 102 37 366.3

(51) Int. Cl. A61L 2/08

(52) U.S. Cl. 522/79

(57) ABSTRACT

The invention relates to a sound-insulating material, especially for automobiles, in the form of a mat, a molding or a coating which is manufactured from rubber and PUR plastic, as well as a method for the manufacture of such a material. In order to lower the weight of the sound-insulating material with essentially unchanged or even improved noise damping effect, in accordance with the invention, it is provided that the rubber (12) and the PUR plastic (13) are mixed with each other, wherein the rubber forms a matrix, in which a plurality of gas-filled, elastic hollow bodies (14) are embedded. The method for the manufacture of the sound-insulating material, in accordance with the invention, is essentially characterized in that thermoplastic rubber particles and PUR plastic particles are extruded together with a foaming agent into a foam-like mix material, wherein the foaming agent is added in the form of microhollow bodies containing foaming agent, which microhollow bodies have a shell of polymer mix that expands by the action of heat. As rubber particles and PUR plastic particles, preferably recycled EPDM rubber or PUR foam pellets are used.

The invention relates to a sound insulating material, especially for automobiles, that is manufactured from thermoplastic rubber and PUR plastic, as well as a method for manufacturing such a material.

In automotive technology heavy layer moldings or heavy layer mats are employed especially for sound insulation in the passenger compartment against engine and driving noises. Moreover, heavy layer moldings and heavy layer mats are used for sound-deadening (body noise damping) of vibrating bodywork. In addition to heavy filler materials the heavy material layers usually contain ethylene propylene diene rubber (EPDM). Amongst others heavy spar barytes (BaSO₄) has proved effective.

Conventional heavy layered material which is employed for sound damping in automobiles, especially for absorbing the sounds of bodywork parts, has a relatively high weight. This is a disadvantage with regard to the attempt of reducing the fuel usage of automobiles by lowering the vehicle weight.

It is the object of the present invention to create a sound-insulating material of the type mentioned in the beginning, especially for automobile construction, which has good soundproofing action and a relatively low weight. Moreover, a cost-effective method for manufacture of such material should be provided.

In regard to the material the solution to this task consists, in accordance with the invention, of mixing the rubber and the PUR plastic with each other, so that the rubber forms a matrix, with a plurality of gas-filled elastic hollow bodies embedded therein.

The method in accordance with the invention is essentially characterized in that thermoplastic rubber particles and PUR plastic particles are extruded accompanied by addition of a foaming agent to a foamed-plastic type mix material, wherein the foaming agent is added in the form of micro hollow bodies containing a foaming agent, said micro hollow bodies have a mixed polymer shell which expands under the influence of heat.

A foamed up heavy layer material is created by the invention, and in fact a heavy layer material with a rubber matrix, that contains expanded elastic hollow bodies. The material in accordance with the invention is characterized by having good sound-proofing as well as sound damping properties and also relatively low weight.

An advantageous development of the invention consists of using recycling material as the rubber particles obtained by comminution of old and/or waste material containing PUR plastic particles. Preferably recycling material can also be used for the PUR plastic particles while old and/or waste material having PUR foaming material is comminuted to pellets or flakes or the like. By doing so raw material resources are spared and raw material costs for the manufacture of heavy layer material are decreased.

Other preferred and advantageous developments of the invention are indicated in the dependent claims.

In the following the invention is further explained on the basis of an embodiment example with reference to the attached drawing. Therein:

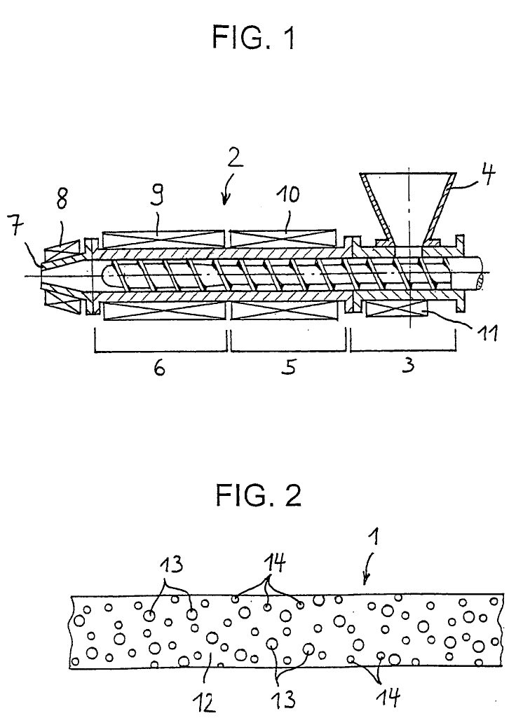

FIG. 1 shows a schematic longitudinal representation of an extrusion device, and

FIG. 2 shows a cross-sectional view of a section of a heavy layer mat manufactured from material in accordance with the invention.

The sound-insulating material in accordance with the invention can have different forms. It can for example be used in the form of a mat, a molding or a manufactured coating by spray casting, especially back injection.

For the manufacture of material in accordance with the invention, that is represented generally as 1 in FIG. 2, an extrusion device 2 is utilized. The extrusion device has essentially the construction shown in FIG. 1. As known per se, the extrusion device 2 has an entry or filling zone 3 with a filling funnel 4, a transition and compression zone 5, a discharge zone 6 and a nozzle 7 as an extrusion tool. The nozzle 7 can be especially arranged as a split nozzle. The split nozzle 7 and the different zones 3, 5, 6 are provided with heating devices 8, 9, 10, 11 which can be controlled independent of each other.

Thermoplastic rubber particles, polyurethane plastic particles and a foaming agent are fed via the filling funnel 4 to the extrusion device 2.

The thermoplastic rubber particles are recycling material in the form of pellets, which are obtained by comminution of EPDM rubber containing material. The EPDM rubber particles fed to the extrusion device 2 preferably have a mean particle size in the range from 2 to 8 mm. The EPDM rubber particles contain heavy spar (BaSO₄) or another heavy filling material.

The PUR plastic particles are likewise preferably recycling material. It is obtained by comminution of PUR foaming material and is in the form of pellets or flakes, which preferably have a mean particle size in the range from 1 to 6 mm.

The thermoplastic rubber particles and the polyurethane plastic particles can for example be obtained by comminuting soundproofing composite structural elements of old automobiles, which are usually formed of a multilayered acoustic spring mass system and have a polyurethane foam layer as acoustic spring and a heavy layer of EPDM rubber as acoustic mass.

As foaming agent, microhollow bodies that contain foaming agent are added, which have a gastight highly elastic shell of mixed polymer and expand under the influence of heat. The microhollow bodies are essentially spherically shaped and in the unexpanded state have an average particle size in the range from 8 to 20 μm and a density in the range from 1000 to 1300 kg/m³.

The foaming agent is a liquid or gaseous hydrocarbon, for example with isobutane. The micro hollow bodies expand through the action of heat, wherein their actual volumes can for example achieve more than 40 times their original volume. The expansion of the microhollow bodies is initiated at a specific temperature. Typical expansion temperatures are in the range from for example 80 to 200° C. The expanded microhollow bodies can be easily compressed and are so elastic, that they are able to withstand several load or pressure changes without their shells bursting.

The heating devices 8, 9, 10, 11 in the extrusion device 2 are controlled so that during the extrusion, in the region of the entry zone 3 there is a temperature of 40 to 50° C., in the region of the transition and compression zone 5 a temperature of 110 to 130° C., in the region of the exit zone 6 a temperature of 120 to 150° C. and in the region of the nozzle 7 a temperature of 120 to 150° C.

The thermoplastic rubber particles, PUR plastic particles and microhollow bodies can be fed to the extrusion device 2 together as a mixture. To avoid de-mixing or as the case may be for the production of an as homogeneous extrusion mass as possible it is if necessary however advantageous, if the thermoplastic rubber particles, PUR plastic particles and the micro hollow bodies containing foaming agent are fed to the extrusion device one after the other in separate charges.

The mixture fed to the extrusion device 2, or as the case may be the extruded material, preferably has the following composition:
70 to 99 wt % thermoplastic rubber particles,
1 to 20 wt % PUR plastic particles, and
0.5 to 10 wt % micro hollow bodies containing foaming agent.

In FIG. 2 a cross-section of a section of extruded material 1 is shown schematically. The thermoplastic EPDM rubber 12 and the PUR plastic 13 are mixed therein with each other essentially homogeneously, wherein the rubber 12 forms a matrix, in which a plurality of expanded, elastic microhollow bodies 14 is embedded. In accordance with the invention, the material 1 therefore consists of foamed heavy layer material that has predominantly closed cells. The density is in the range of 0.2 to 1.5 kg/cm³. Preferably the density of material 1 is less than 1.0 kg/cm³, and especially preferred less than 0.5 kg/cm³. Conventional unfoamed heavy layer material on the other hand usually has a density of bout 1.8 kg/cm³.

1. A sound-insulating material (1), especially for automobiles, manufactured from rubber (12), that is mixed with PUR plastic (13) wherein the rubber forms a matrix, in which a plurality of gas-filled hollow bodies (14) are embedded, wherein the rubber (12) is formed from thermoplastic rubber particles.

2. The sound-insulating material according to claim 1, wherein the rubber (12) and/or the PUR plastic (13) is a recycling material.

3. The sound-insulating material according to claim 1, wherein it is composed of
70 to 99 wt % rubber (12),
1 to 20 wt % PUR plastic (13)
0.5 to 10 wt % gas-filled hollow bodies (14).

4. The sound-insulating material according to claim 1, wherein the rubber (12) is an EPDM rubber.

5. The sound-insulating material according to claim 1, wherein the gas-filled hollow bodies (14) have a shell of mixed polymer.

6. The sound-insulating material according to claim 1, wherein it has a density of less than 1.5 kg/dm³ or g/cm³, preferably less than 1.0 kg/dm³ or g/cm³.

7. A method of manufacturing a sound-insulating material (1), especially for automobiles, wherein the thermoplastic rubber particles and PUR plastic particles are extruded, while adding a foaming agent to a foam-like mix material, wherein the foaming agent is added in form of foaming agent containing micro-hollow bodies (14), which have a shell of polymer mix and expand during heat treatment.

8. The method according to claim 7, wherein as thermoplastic rubber particles recycling material is used which is obtained by comminution of material containing EPDM rubber.

9. The method according to claim 7, wherein as PUR plastic particles recycling material is used that is obtained by comminution of material containing PUR foamed material.

10. The method according to claim 7, wherein referred to the sound-insulating material (1) to be manufactured, 70 to 99% wt % thermoplastic rubber particles, 1 to 20 wt % PUR plastic particles, and 0.5 to 10 wt % microhollow bodies containing foaming agent are fed to an extrusion device (2).

11. The method according to claim 7, wherein microhollow bodies (14) containing the foaming agent are spherically shaped.

12. The method according to claim 7, wherein the thermoplastic rubber particles, the PUR plastic particles and the microhollow bodies containing the foaming material are fed to the extrusion device in separate charges.

13. The method according to claim 7, wherein extrusion device (2) has an entry zone (3), a transition and compression zone (5) and an exit zone (6) with a nozzle (7) following thereon and is heated such that during extrusion the following temperatures exist:
40 to 50° C. in the region of the entry zone,
110 to 130° C. in the region of the transition and compression zones,
120 to 150° C. in the region of the exit zone, and
120 to 150° C. in the region of the nozzle.
